(19) United States
(12) Patent Application Publication
(10) Pub. No.: US 2024/0009014 A1
(43) Pub. Date: Jan. 11, 2024

(54) THREE-DIMENSIONAL ORTHOSES HAVING MULTIPLE ADJUSTMENT FEATURES AND METHODS FOR THEIR MANUFACTURE AND USE

(71) Applicant: Osteoid Saglik Teknolojileri A.S., Istanbul (TR)

(72) Inventor: Deniz Karasahin, Istanbul (TR)

(73) Assignee: Osteoid Saglik Teknolojileri A.S., Istanbul (TR)

(21) Appl. No.: 18/473,089

(22) Filed: Sep. 22, 2023

Related U.S. Application Data
(63) Continuation of application No. PCT/IB22/00167, filed on Mar. 23, 2022.
(60) Provisional application No. 63/166,186, filed on Mar. 25, 2021.
(60) Provisional application No. 63/167,758, filed on Mar. 30, 2021.

(51) Int. Cl. A61F 5/01; A61F 5/058

(52) U.S. Cl. CPC A61F 5/0102; A61F 5/05841; A61F 2005/0167

(57) ABSTRACT

A conformable body interface is fabricated using a data set representing a three-dimensional, soft tissue body surface. The conformable body interface includes a body scaffold that is divided into two or more longitudinal segments separated by axial joints. Optionally, the body scaffold is further divided into two or more circumferentially split segments separated by circumferential joints. The axial joints are circumferentially constrained by elastic bands, tabs, or similar structures and the circumferential joints are longitudinally constrained by elastic axial tethers or similar structures. In this way, the body interfaces can accommodate swelling and bending of the body surface.

### CROSS-REFERENCE TO RELATED APPLICATIONS

This application is a continuation of PCT Application No. PCT/IB22/000167, (Attorney Docket No. 50016-705.601), filed Mar. 25, 2022, which claims the benefit of U.S. Provisional No. 63/166,186 (Attorney Docket No. 50016-705.101), filed Mar. 25, 2021, and of U.S. Provisional No. 63/167,758 (Attorney Docket No. 50016-705.102), filed Mar. 30, 2021, the entire contents of which are incorporated herein by reference.

### BACKGROUND OF THE INVENTION

### 1. Field of the Invention

The present invention relates generally to medical devices and their methods of use and manufacture. More particularly, the present invention to personalized orthoses with features to permit shape modification over time as the patient's condition changes.

While orthotic intervention for physical rehabilitation has been known for centuries, splints, casts, and other orthoses still present challenges in implementation. Starting with temporary immobilization using splints made from sticks and casts made from plaster, the field has progressed to the production of “3D printed” orthoses, where orthoses are fabricated for individual patient anatomies by scanning a patient anatomy to generate a three-dimensional digital representation of that anatomy, using computer-aided design (CAD) tools to produce a digital file representing an individualized orthosis, and fabrication of the orthosis using 3D printing techniques.

Although 3D printed orthoses are more comfortable and hygienic than traditional plaster casts, they fail in practicality due to complicated workflow and many hours of 3D printing required for each individual patient who is also in need of urgent medical assistance. In particular, orthoses configured for a patient at an early stage of treatment may not provide a proper corrective fit during subsequent stages of recovery, requiring significant modification or in some instances fabrication of a replacement orthosis.

An orthosis which adapts to changes in the patient's anatomy during healing is described in WO2016/170433, commonly owned with the present application. As shown in FIG. 1, the orthosis of WO2016/170433 consists of a body scaffold 100 divided axially along circumferential lines 101 and circumferentially along axial lines 102, resulting in a plurality of structurally separate cells 110, one of which is broken out in FIG. 1. As best seen in FIGS. 2 and 3, circumferentially adjacent cells 110 are joined by slip joints 201 configured to expand along paths 202 held together by elastic constraints 103 while axially adjacent cells are held together by axial tethers 104. The orthosis may be removably placed over a selected portion of human anatomy, for example an arm and a portion of a hand 106, as seen in FIG. 1. The cells 110 can be temporarily separated to facilitate placement and removal.

While a significant advance in the art, the orthosis design of WO2016/170433 has certain limitations. For example, the axial slip joints can only accommodate a restricted path of motion, limiting the orthosis to a single volumetric change pattern which in turn limits the types of cases in which it can be used. As seen in FIGS. 2 to 5, such limitation arises from changes in the tangent vectors between the male and female portions of the axial slip joints 201 as the circumference of the arm or other anatomy changes. An increase in circumference results in a flatter (greater radius) tangent vector 203 (FIG. 3) while a decrease in circumference results in a less flat (smaller radius) tangent vector 204 between axial slip joints (FIG. 4). Other volumetric changes can result in an uneven distribution of stress between slip joints 205 (FIG. 5).

For these reasons, it would be desirable to provide improved and alternative orthotic designs which can accommodate a wide variety of both predictable and unpredictable changes in patient anatomy during treatment and healing after an initial design and fabrication of the orthosis has been completed. At least some of these objectives will be met by the inventions described and claimed herein.

### 2. Listing of Background Art

Related publications include commonly owned US2021/0205115; US2017/0224520; WO2016/170433; and WO2016/071773, the full disclosures of which are incorporated herein by reference.

### SUMMARY OF THE INVENTION

The present invention provides orthoses having an improved ability to accommodate a wide variety of both predictable and unpredictable changes in patient anatomy during treatment and healing. While particularly suitable for “individualized” orthoses made by 3D printing from scans of an injured patient, the designs and methods of the present invention will also find use in the design and fabrication of “off the shelf” orthoses which may be molded or otherwise fabricated to a “standard” size and anatomy and thereafter be size-adjusted for particular patients at the outset of treatment. In all cases, the orthoses of the present and have the ability to be further adjusted during the course of treatment.

The orthoses of the present invention will typically have pivotally attached circumferential slip joints which accommodate changes in a circumferential shape of the patient anatomy, such as an arm, leg, joint or appendage, during healing. Other mechanical features that may be introduced to enhance accommodate to changes in anatomy during the course of healing include radial spline locks and worm drives for re-stabilizing the body scaffold. The orthoses of the present invention will preferably also include features for axial length adjustment and three-dimensional alignment between circumferentially and axially split cells in the orthosis. Specific mechanical features that may be incorporated into the orthoses of the present invention include a spindle and rotary ratchet mechanism for locking, releasing, and adjusting the tension of the axial tethers. Additional features that may be incorporated into the orthoses of the present invention include auxetic structural elements spanning circumferentially and/or axially split cells to ease assembly and fitting sessions.

In a first aspect, the present invention provides a conformable orthosis comprising a scaffold having a longitudinal axis and being configured to be removably placed over a body surface. The body scaffold is divided into a plurality of scaffold cells, each cell having a first side, a second side, a top, and a bottom, wherein a first side on one scaffold cell and a second side on a circumferentially adjacent scaffold cell are separable along an axial line and the top and bottom on axially adjacent scaffold cells are separable from each other along circumferential lines. A first circumferential connector is pivotally attached to the first side of at least some of the scaffold cells and a second circumferential connector pivotally attached to the second side of at least some of the scaffold cells, and the first and second circumferential connectors are configured to detachably connect at an adjustable distance therebetween. In this way the tops and bottoms on axially adjacent scaffold cells are adjustably coupled to each other.

In some instances, the conformable orthoses of the present invention may further comprise axial tethers which adjustably couple the tops and bottoms on axially adjacent scaffold cells. For example, the axial tethers may pass axially through a pivotal axis of at least some of the first and second circumferential connectors.

In some instances, the conformable orthoses of the present invention may further comprise at least one uptake spindle for adjustably tightening one or more axial tethers. For example, the uptake spindles comprise a rotary ratchet and pawl mechanism.

In some instances, the conformable orthoses of the present invention may further comprise spacers configured to be placed between the tops and bottoms of axially adjacent scaffold cells.

In some instances, axially adjacent scaffold cells of the conformable orthoses of the present invention may be connected by threaded fasteners. For example, the threaded fasteners may be incorporated into ball and socket joints to permit realignment of the axially adjacent scaffold cells.

In some instances, the first and second circumferential connectors of the conformable orthoses of the present invention may comprise rotating spindles with radially extending coupling tabs. For example, the coupling tabs of the first and second circumferential connectors may be configured to detachably lock with the coupling tabs of the second and first circumferential connectors, respectively, of circumferentially adjacent scaffold cells. At least some of the radially extending coupling tabs may have grooved surfaces configured to be locked with grooved surfaces on coupling tabs on adjacent scaffold cells.

In some instances, the rotating spindles of the conformable orthoses of the present invention may have upper and/or lower radially splined surfaces configured to selectively lock with the scaffold cell.

In some instances, the first and second circumferential connectors of the conformable orthoses of the present invention may further comprise driving screws configured to engage rotary gears on a periphery of at least some of the rotating spindles to rotate the spindles to adjust an angle of the coupling tabs.

### BRIEF DESCRIPTION OF THE DRAWINGS

FIG. 1 illustrates a prior art adaptive splint with twelve separable cells split along four circumferential lines and three axial lines held together with three elastic circumferential restraints and four axial tethers as described in WO2016170433.

FIG. 2 is a transverse cross-sectional view taken along line 1-1 of FIG. 1 of the splint of FIG. 1 shown with four slip joints in a circumferentially expanded configuration.

FIG. 3 is a detailed view of one of the slip joints of FIG. 2 shown in a closed configuration illustrating one pre-defined path of motion.

FIG. 4 is a view similar to that of FIG. 3 illustrating the change in tangent vector of the slip joint due to circumferential expansion.

FIG. 5 is a view similar to that of FIG. 3 showing the slip joint in a partially opened configuration illustrating the internal imbalances due to change in tangent vector due to circumferential expansion.

FIG. 6 is a schematic illustration of a pivoting circumferential connector intended to form a slip joint in combination with a mating connector (not shown) in accordance with the principles of the present invention.

FIG. 7 is a perspective view of a scaffold cell having first and second circumferential connectors configured to couple with mating connectors on circumferentially adjacent scaffold cells to form pivoting radial slip joints.

FIG. 8 is a schematic transverse cross-section of an orthosis of the present invention in radial expansion with pivoting slip joints in circumferentially expanded configuration.

FIG. 9 is an embodiment of an extra adaptive wrist splint with four longitudinally split cells and three circumferentially split cells comprising a total of twelve individual body scaffold cells held together with pivoting slip joints with radial gears and four axial tethers. An axially split cell of the extra adaptive splint is highlighting three individual body scaffold cells held together with three male type pivoting circumferential connectors with radial gears facing each other, three female type pivoting circumferential connectors with radial gears facing each other and an axial tether looping through the split cell.

FIG. 10 is an exploded view of a split cell with one male type pivoting circumferential connector with radial spline locking surfaces and one female type pivoting circumferential connector with radial spline locking surfaces.

FIG. 11 illustrates two axially adjacent scaffold cells with two removably placed spacers between cells, increasing the length of the axial segment.

FIG. 12 is a perspective view of a scaffold cell having two pivoting circumferential connectors with worm drives.

FIG. 13 is an exploded view of the scaffold cell of FIG. 12.

FIG. 14 is an embodiment of an extra adaptive ankle splint with 14 individual body scaffold cells held together with pivoting slip joints with radial gears, 6 threaded fasteners (4 visible) and four axial tethers spanning across the body scaffold.

FIG. 15 a detailed view of the extra adaptive ankle splint of FIG. 14 with portions broken away.

FIG. 16 is an exploded view of the extra adaptive ankle splint of FIG. 15.

FIG. 17 is an embodiment of an extra adaptive ankle splint with 14 individual body scaffold cells held together with pivoting slip joints with radial gears, six threaded fasteners (four visible), four threaded fasteners with two ball joints (two visible), and four axial tethers spanning across the body scaffold.

FIG. 18 is a detailed view of a threaded fastener with two ball joints and adjacent scaffold cells of the extra adaptive ankle splint of FIG. 17.

FIG. 19 is an exploded view of a threaded fastener with ball joints.

FIG. 20 illustrates the threaded fastener of FIG. 19 in a closed configuration.

FIG. 21 illustrates the threaded fastener of FIG. 19 in a partially opened configuration.

FIG. 22 is an exploded view of a threaded fastener with a ball joint with a conical screw and an expanding sphere as ball joint.

FIG. 23 is an exploded view of a threaded fastener having two ball joint mechanism with collets.

FIG. 24 illustrates an axially and circumferentially split cell with a rotary ratchet 701 attached to an axial tether with two pivoting circumferential connectors.

FIG. 25 shows the assembly of FIG. 24 with the rotary ratchet 701 shown in exploded view.

FIG. 26 illustrates portions of the rotary ratchet 701 of FIGS. 24 and 25 shown in an exploded side view.

FIG. 27 illustrates an embodiment of an extra adaptive splint with four longitudinally split segments and four circumferentially split segments comprising a total of sixteen individual body scaffold cells held together with pivoting slip joints with radial gears and four axial tethers and four rotary ratchets. An axially split segment of the extra adaptive splint is highlighting four individual body scaffold cells held together with four male type pivoting slip joints with radial gears facing each other, four female type pivoting slip joints with radial gears facing each other and an axial tether looping through the split segment.

FIG. 28 is an exploded view of four axial body scaffold cells comprising an axially split cell of the body scaffold of FIG. 27, with four male type pivoting connectors with radial gears and four female type pivoting connectors with radial gears with seven further divided scaffold cell assemblies with radial spline locking surfaces, with one further one divided scaffold cell assembly with radial spline locking surfaces and embedded rotary ratchet, four male type 711 and four female type 712 pivoting circumferential connectors with radial spline locks, one rotary ratchet mechanism and an axial tether coupled with a rotary ratchet.

FIG. 29 illustrates a pivoting and clamping circumferential slip joint with radial spline surfaces expanded configuration from front view, illustrating the passing of axial theders through the mechanism in thus illustrating the principles of the clamping mechanism.

FIG. 30 illustrates one pivoting and clamping circumferential male connector with radial spline surfaces and one a pivoting and clamping circumferential female connector with radial spline surfaces from front view.

FIG. 31 illustrates one pivoting and clamping circumferential male connector with radial spline surfaces and one a pivoting and clamping circumferential female connector with radial spline surfaces in perspective view. Emphasizing on the slot passing through the male connector.

FIG. 32 illustrates a conformable orthosis having an auxetic geometry spanning across a circumferentially and axially split body scaffold cell in expanded configuration. Pivoting circumferential connectors and axial theaters are also incorporated according to the principles of the invention.

FIG. 33 illustrates a conformable orthosis having an auxetic geometry spanning across a circumferentially and axially split body scaffold cell in closed configuration. Pivoting circumferential connectors and axial theaters are also incorporated according to the principles of the invention.

### DETAILED DESCRIPTION OF THE INVENTION

The present invention utilizes known techniques in digital manufacturing and computer aided design to provide exo-skeletal orthosis configurable to accommodate unpredicted changes in patient anatomy in the form of medical casts with a plurality of adjustable and self-adjusting structural components. The structural solutions of the invention are developed to promote a controlled and medically beneficial relationship between the orthotic and the patient. While particularly suitable for digital design and fabrication of orthoses, the adjustable features and designs of the present invention are also useful in the design and fabrication of “off the shelf” orthoses which may be molded or otherwise fabricated to a “standard” size and anatomy and thereafter be size-adjusted for particular patients at the outset of treatment.

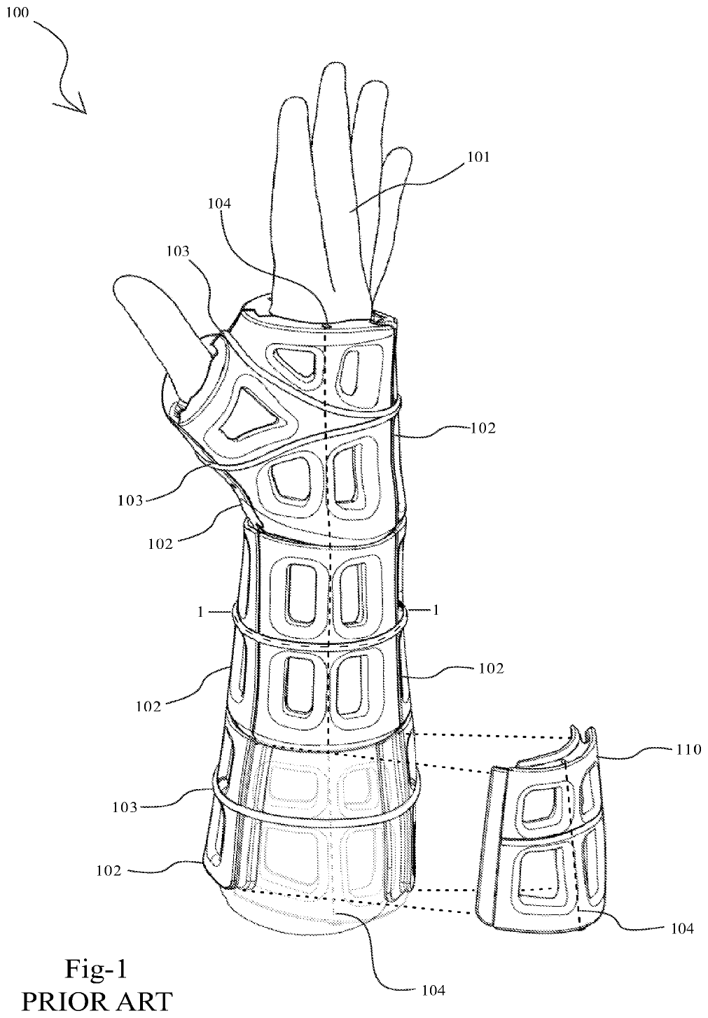

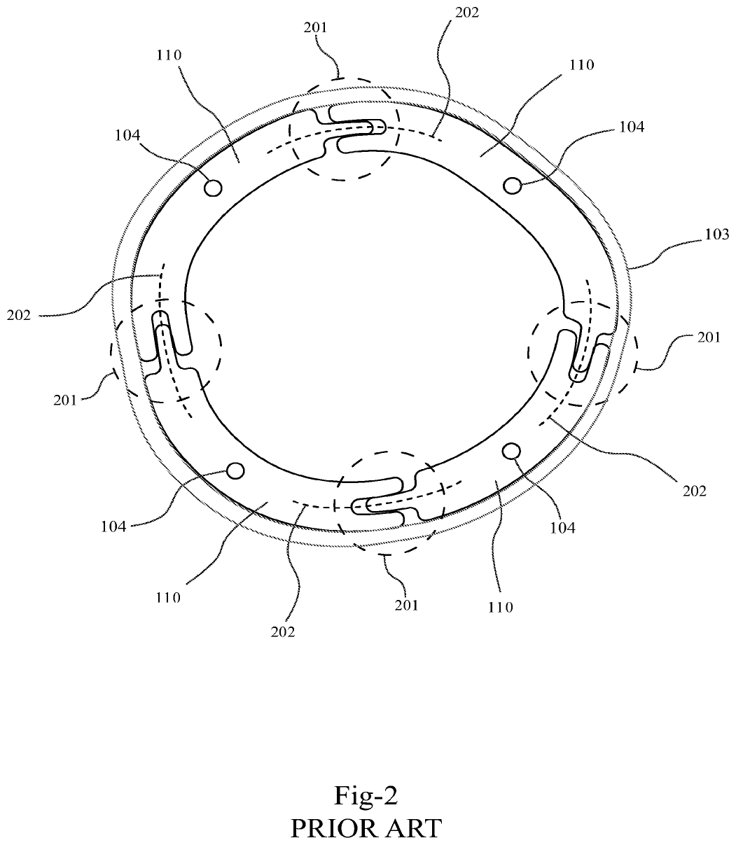

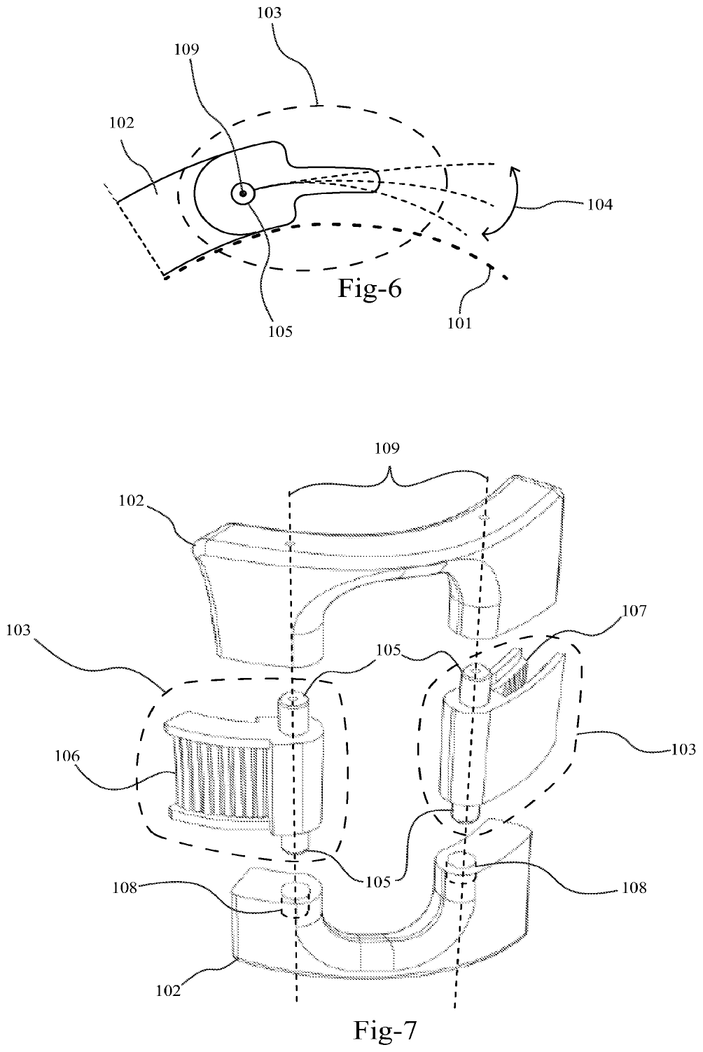

The limiting factor described in detail in the background of the invention (FIGS. 1 to 5) due to changes in tangent vector, has been overcome with the introduction of an additional degree of freedom 104 to the circumferentially expanding split cell in the form of rotating pivots aligned at the back of each slip joint. Thanks to the pivots the geometric changes caused by the variation in different tangent vectors is not a limiting factor anymore. FIGS. 6, 7 and 8 illustrate the methods for incorporating pivoting slip joints.

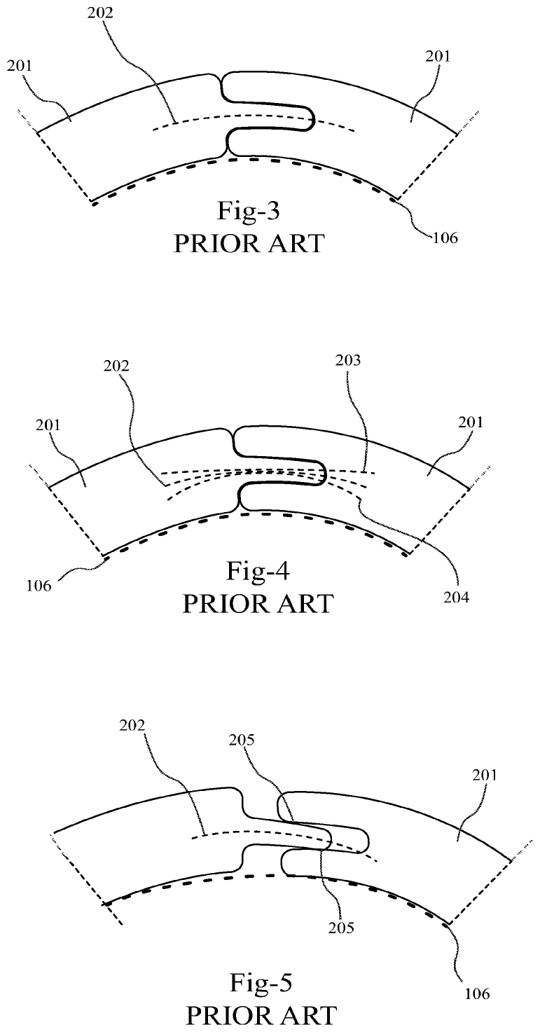

FIG. 6 illustrates a schematic illustration (in similar fashion with cross sections from FIGS. 3, 4 and 5) of an axially and circumferentially split scaffold cell 102 with a pivoting circumferential connector 103. The pivot is 105 aligned at the back of the circumferential connector 103. The pivot in this case is providing the additional degree of freedom to the circumferentially expanding structure over a soft tissue of human anatomy 101.

FIG. 7 is a three-dimensional representation of an axially and radially split scaffold cell 102 with two pivoting circumferential connectors 103. Each pivoting circumferential connector is configured to couple with neighboring pivoting circumferential connectors via interlocking geometries with either male 106 or female 107 configuration. The scaffold cell is also modified to incorporate fitting sockets 108 for the pivots. The axial pivots also allow the passing of axial tethers 109 spanning across their length.

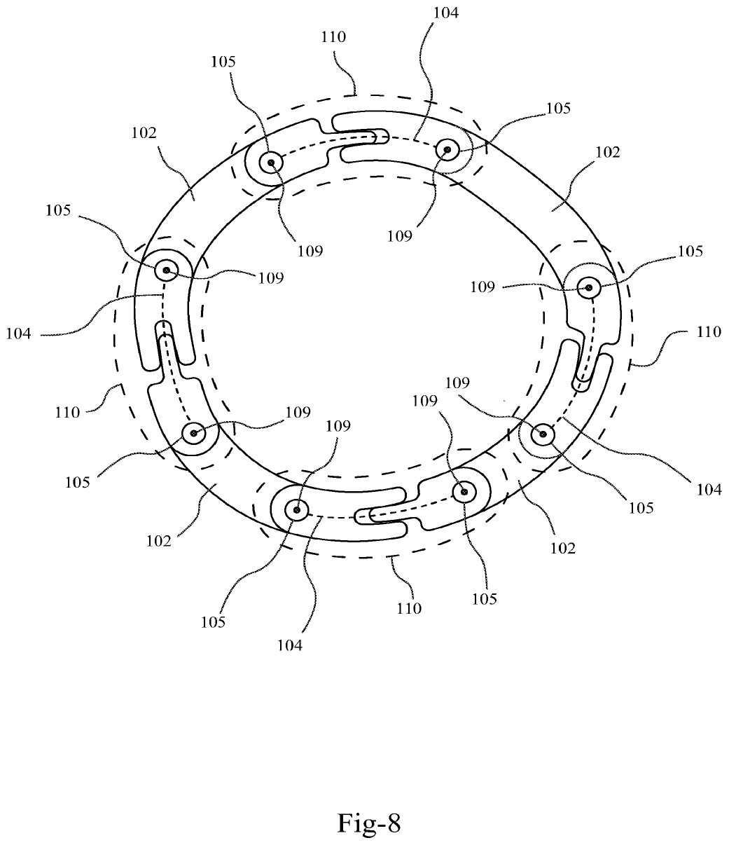

FIG. 8 is a schematic cross-section on an orthosis of the present invention in radial expansion with pivoting slip joints in circumferential expansion. 4 Slip joints 110, 8 pivots 105 and 8 axial tethers 109 are illustrated.

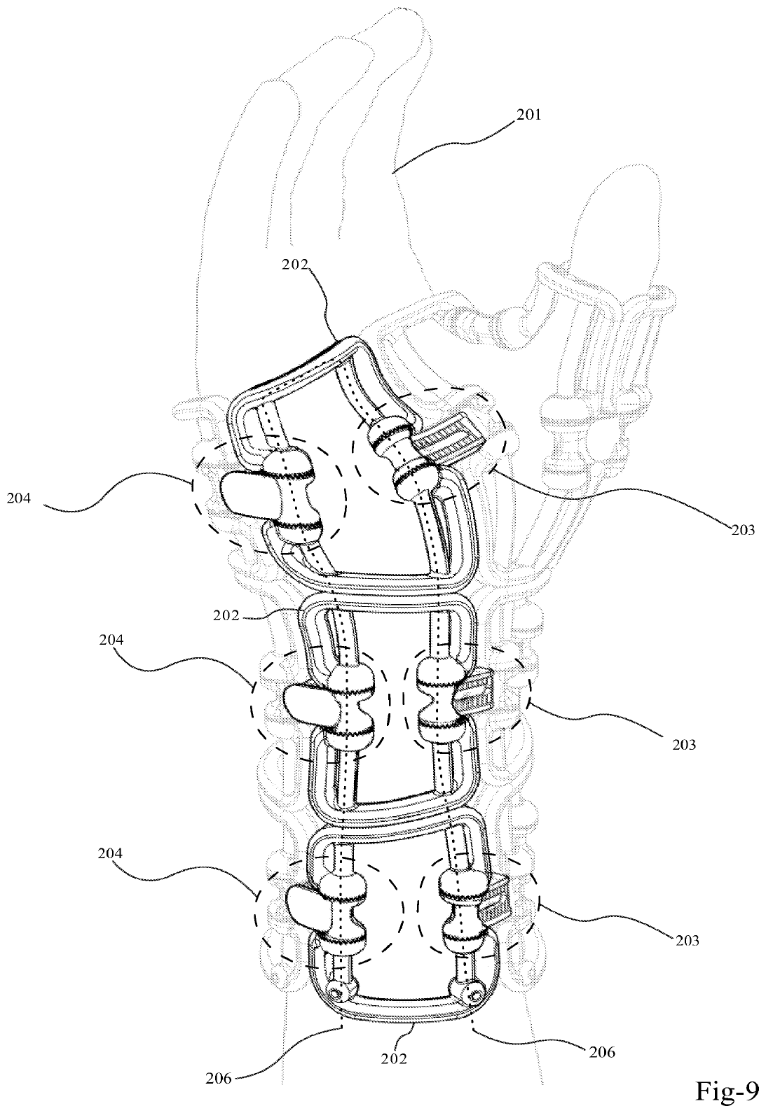

Although the additional degree of freedom 104 overcomes the changing tangent vector limitation, which unbalances the structural rigidity if left un-constrained. Present invention provides methods to constrain pivoting circumferential connectors and re-stabilizing the structure. The methods are illustrated between FIGS. 9-15.

In an embodiment demonstrated between FIGS. 9-11, pivoting circumferential connectors are constrained with the use of radial spline locking surfaces. The radial spline locking surfaces are critically positioned between pivoting circumferential connectors and further divided split scaffold cells comprising radial gears. Axial tethers are also passing through the split scaphoid cells and pivoting circumferential connectors in accordance with the principles of the invention.

FIG. 9 is an embodiment of an extra adaptive orthosis with four longitudinally split segments and three circumferentially split segments comprising a total of twelve individual body scaffold cells held together with pivoting slip joints with radial gears and four axial tethers. The extra adaptive splint in this embodiment is placed over a wrist 201. An axially split segment of the extra adaptive splint is highlighting three individual body scaffold cells 202 comprising an axial segment is held together with three male type pivoting circumferential connectors with radial spline locks 203, three female type pivoting circumferential connectors with radial spline locks 204 and an axial tether 206 is looping through the embodiment 206.

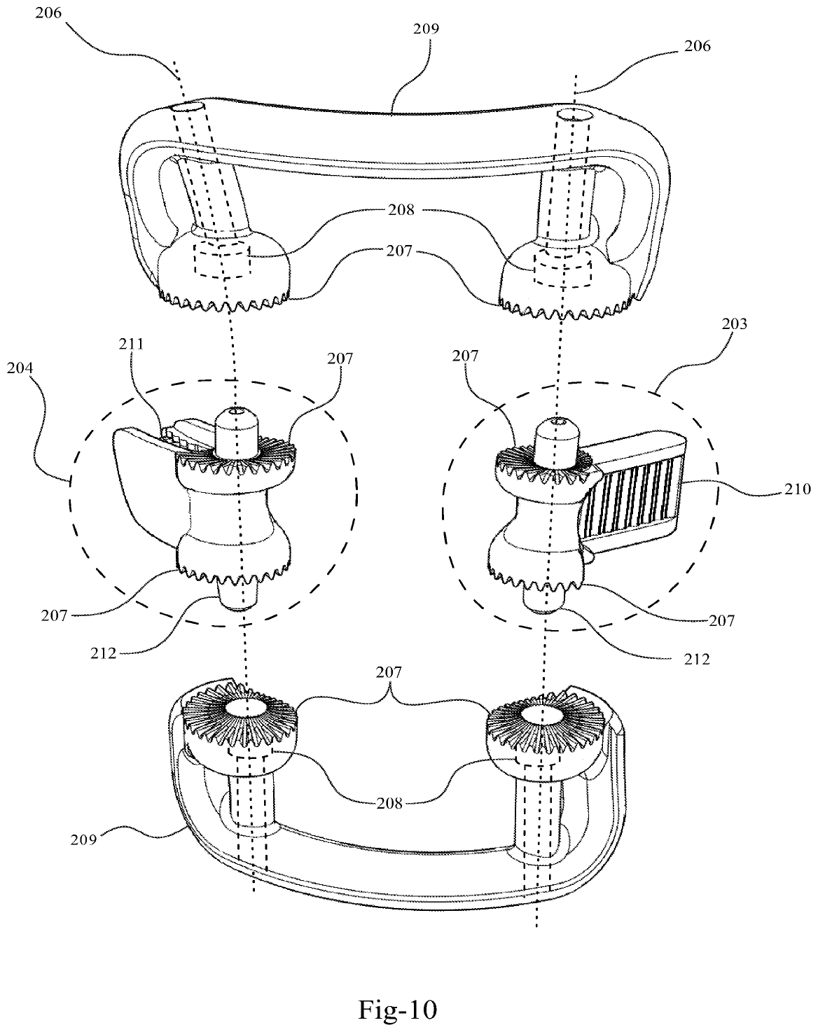

FIG. 10 is a blowup demonstration of a single axially and radially split scaffold cell of the body scaffold described in FIG. 9. Two further divided scaffold cell assemblies with radial spline locking surfaces 209, are aligned at the top and bottom of pivoting circumferential connectors. The circumferential connectors are held by the sockets 208 located in scaffold cells. One male type pivoting circumferential connector with radial spline locking surfaces 203 comprising male type circumferential connector 210, axial pivot 212 and radial spline locks 207. One female type pivoting circumferential connector with radial spline locking surfaces 204 comprising female type circumferential connector 211, axial pivot 212 and radial spline locks 207.

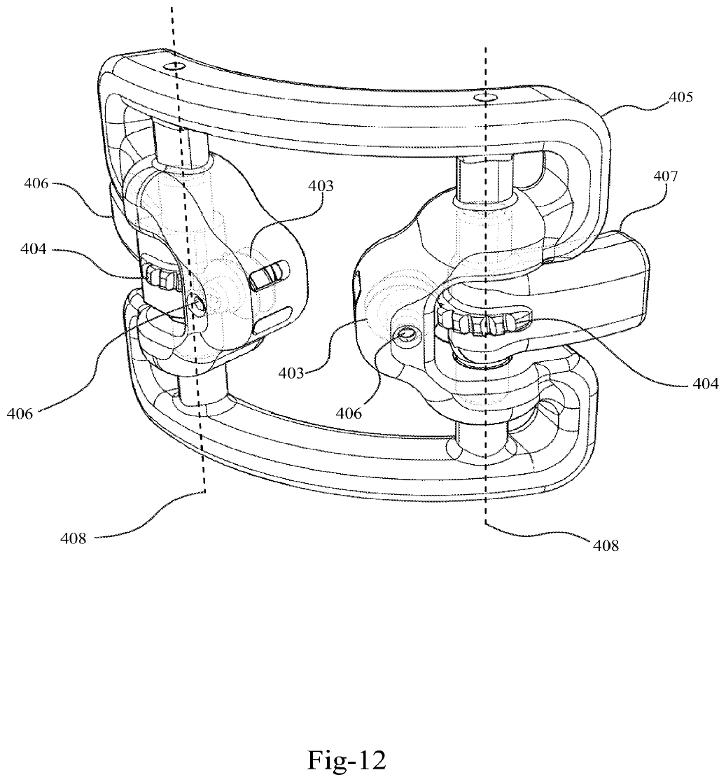

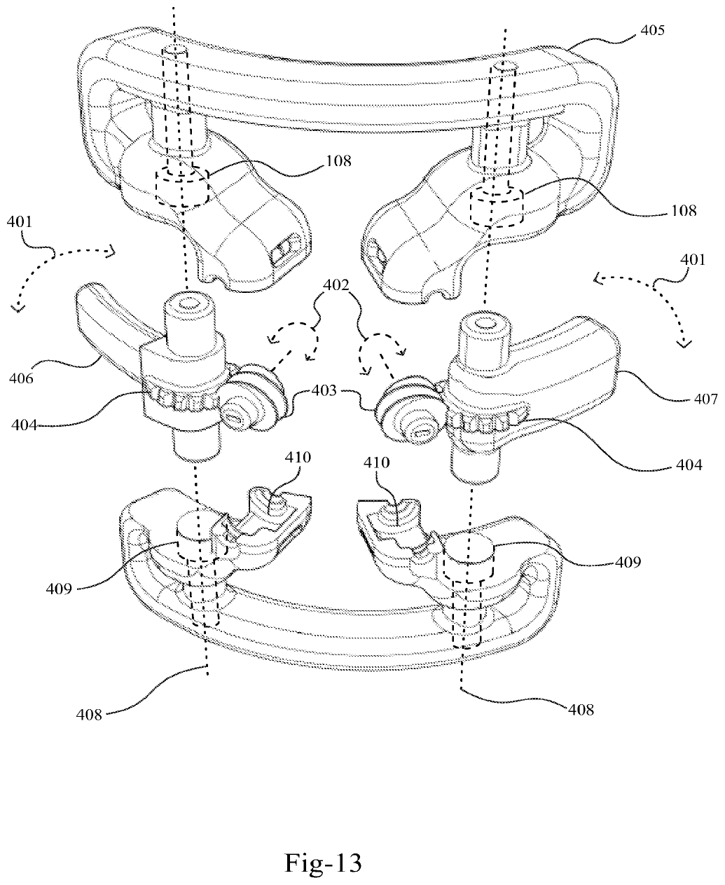

In another embodiment demonstrated between FIGS. 12 and 13, the positioning of pivoting circumferential connectors is controlled by adapting a worm drive mechanism to pivoting circumferential connectors. The worm drive mechanism consists of a helically threaded (snail) shaft 403 which engages a worm wheel 404 so that turning the snail shaft rotationally positions a male connector 406 or female connector 407. The worm drive determines the position 401 of pivoting circumferential connectors, by rotating 402 the worm shaft 403 thus pivoting the circumferential connectors. The worm drives positioned inside the scaffold cell are accessible through openings 406 in the design. The advantage of using the worm drive is the ability to autoblock.

FIG. 12 illustrates an axially and radially split scaffold cell 405 of a worm drive enabled pivoting circumferential connector. One worm drive enabled pivoting circumferential male connector 406 and one worm drive enabled pivoting circumferential female connector 407 are visible in the illustration. Also, normally hidden snail shafts 403 are demonstrated in ghosted illustration. Axial tethers 408 are passing through the scaffold cell 405 in accordance with the principles of the invention.

FIG. 13 is an exploded view of the structure in FIG. 12, illustrating a single axially and radially split scaffold cell. This illustration further demonstrates the sockets for worm drive enabled pivoting circumferential connectors 409 and socket for worm shafts 410.

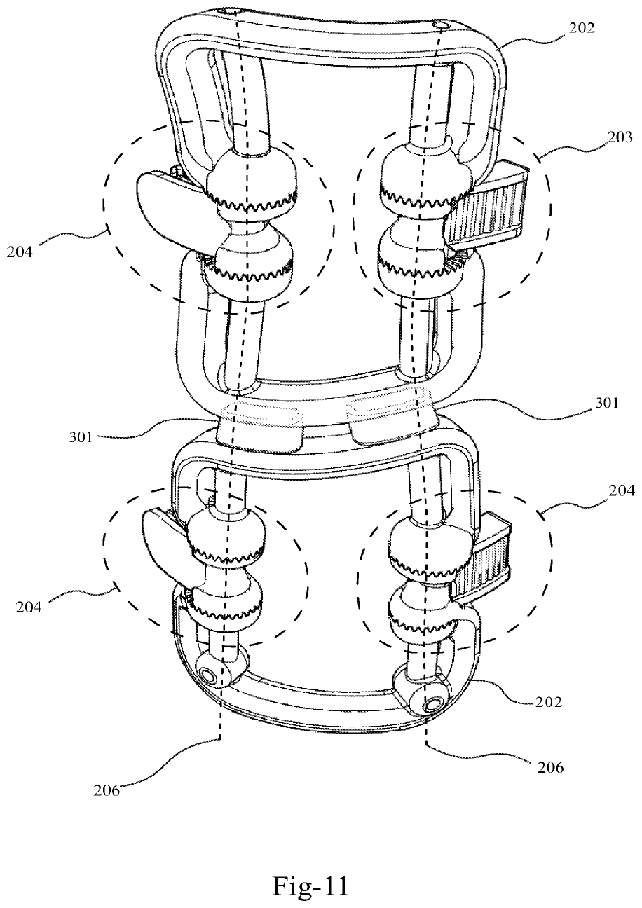

Length is an equally important parameter as circumference for many types of orthoses. Present invention provides methods for axial length adjustment between circumferentially and axially split cells. In its first aspect illustrated in FIG. 11, the method comprises inserting removably placed separators 301 to snap fit between axially spaced apart cells 202 to provide a stable support thus expanding the axial length of the structure with pre-determined geometries.

FIG. 11 demonstrates two axially spaced apart split scaffold cells with two removably placed separators 301 between them, adjusting the length of the axial segment by placing or removing the separators.

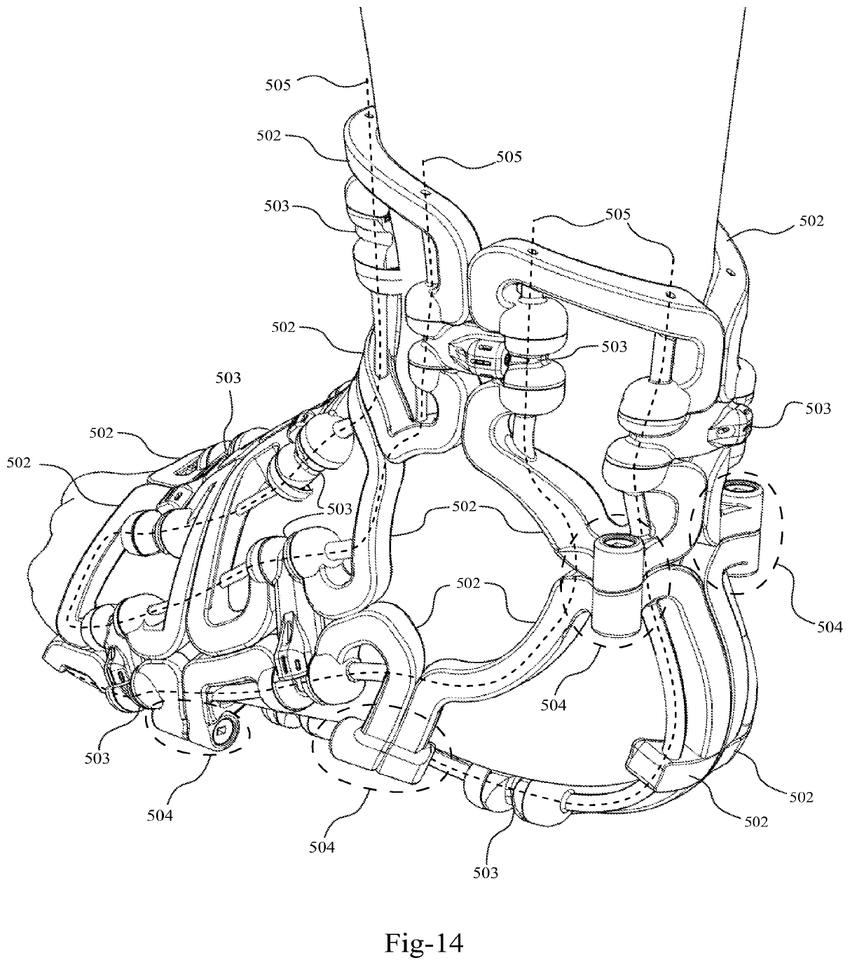

In its second aspect precise control over the axial length of the body scaffold can be configured by using linear actuator type mechanisms between axially spaced apart scaffold cells. In an embodiment described in FIGS. 14, 15 and 16 threaded fasteners are placed between split cells of a lower extremity orthosis using embedded bolts and fitting threaded sockets. The length is determined by rotation of embedded bolts.

FIG. 14 is an embodiment of an extra adaptive ankle splint with fourteen individual body scaffold cells held together with pivoting slip joints with radial gears 503, six threaded fasteners 504 (four visible) and four axial tethers 505 spanning across the body scaffold in accordance with the principles of the invention.

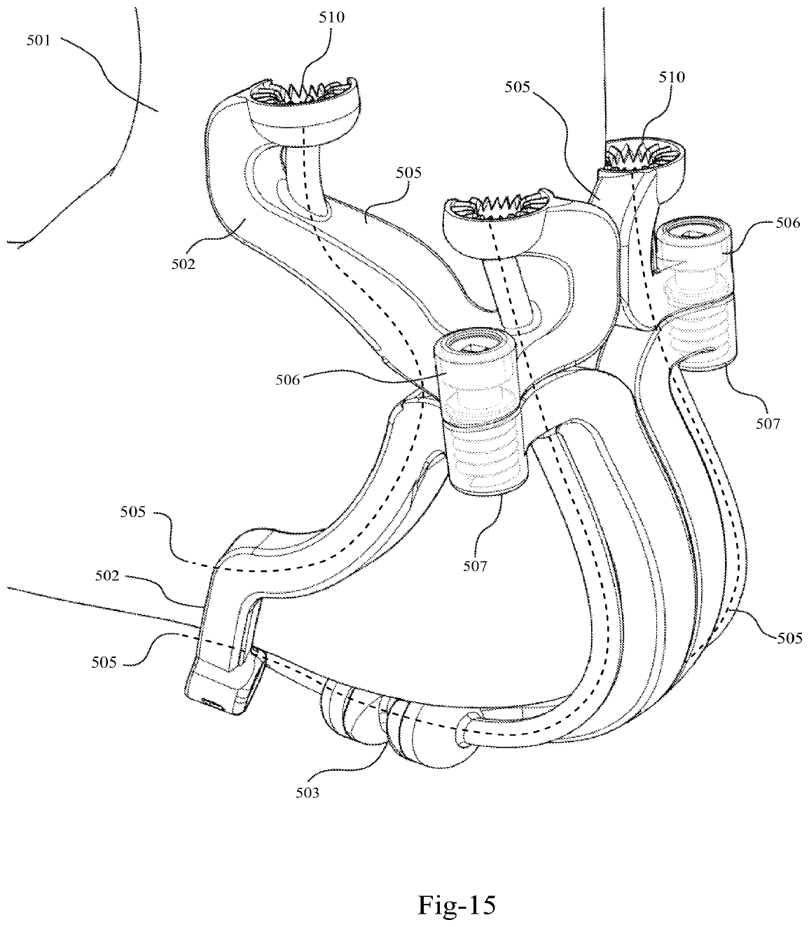

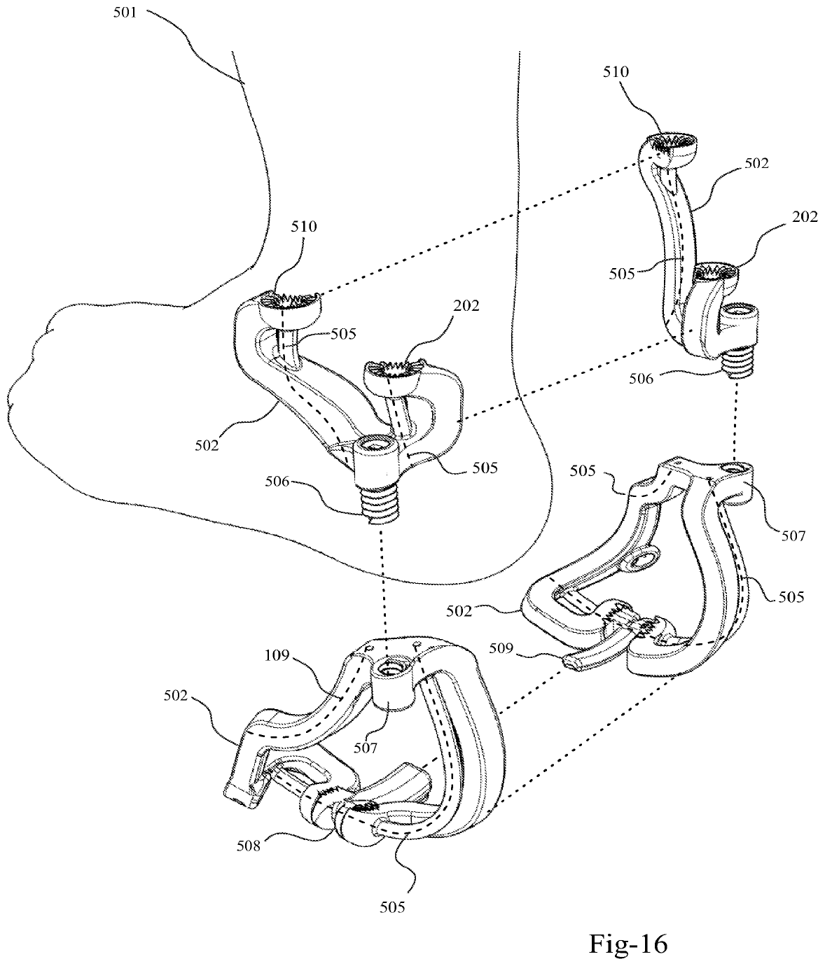

FIGS. 15 and 16 demonstrate the same extra adaptive ankle splint of FIG. 14 focusing on the components corresponding to the Achilles tendon of the patient. Embedded bolts are illustrated with 506 the corresponding threaded sockets illustrated with 507 for providing more detail.

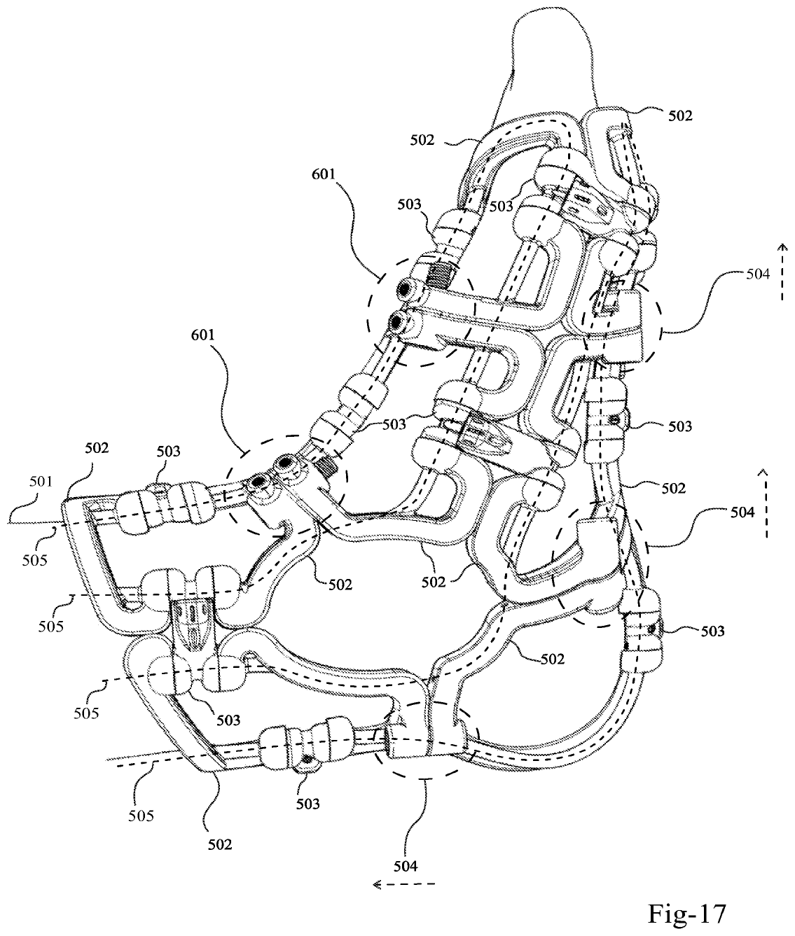

Lower extremity orthoses are also challenging because of the bending geometry in the metatarsal, particularly at the posterior side. The problem is accommodating axial expansion between two points of varying positions due to changes in the expansion geometries. Although the problem is similar to tangent vector limitations, which is a 2D alignment problem, this one is a 3D alignment problem. FIG. 17 illustrates multiple axial expansion mechanisms with their vectors of expansion, which are perpendicular to each other in this case. When it is considered that the slip joints also have their own paths of motion, three-dimensional alignment between split scaffold cells is required to overcome the problem.

In an embodiment described between FIGS. 17 and 21 of the present invention, the threaded fasteners are combined with ball and socket joints to allow 3D alignment between axially split scaffold cells. Methods are further improved with constraints to the ball and socket joints, allowing further adaptability and stabilization to the body scaffold.

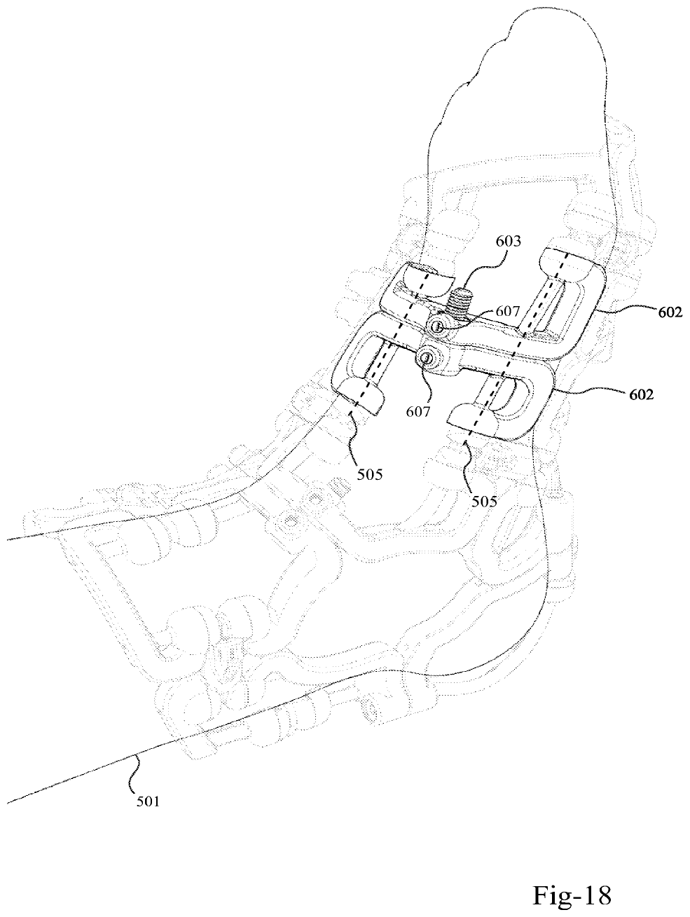

FIG. 18 is highlighting a threaded fastener with ball and socket joints and two axially adjacent split scaffold cells of the extra adaptive ankle splint, corresponding to the posterior metatarsal region of the ankle.

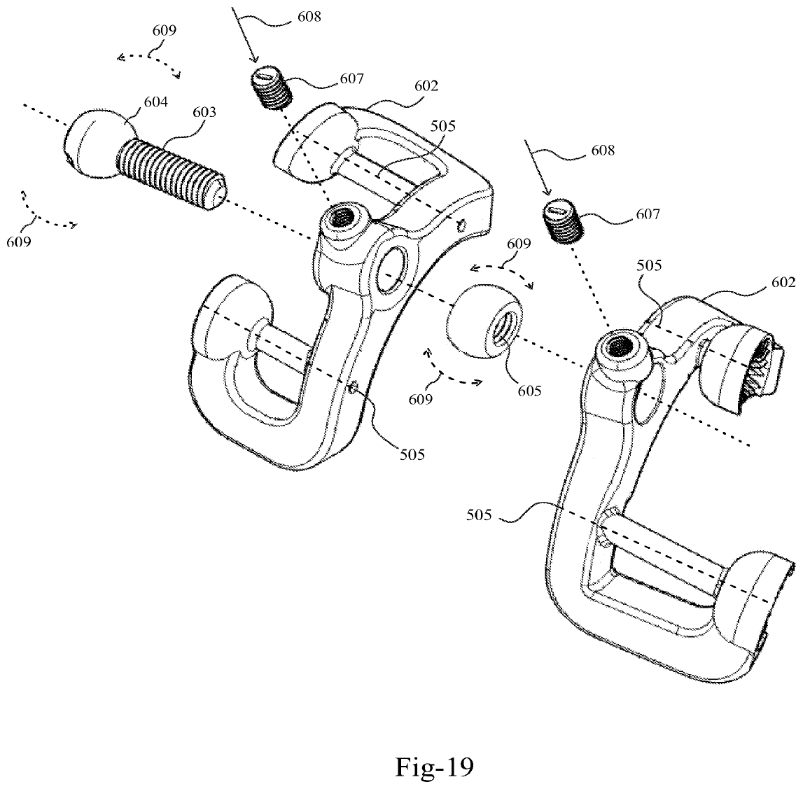

FIG. 19 is providing greater detail to the threaded fastener with ball and socket joints, and two adjacent divided scaffold cells of an extra adaptive ankle splint in blow up demonstration. The bolt 603 section of threaded connector is modified to incorporate a sphere 604 to allow 3D alignment 609. The further divided scaffold cell assemblies with radial spline locking surfaces 602 are modified to incorporate a spherical socket. The threaded socket 605 of threaded fastener is also modified to incorporate a spherical outer shell to allow 3D alignment 609. The constraints for stabilizing the structure is in the form of a locking mechanism consisting of set screws 607 and perpendicularly aligned threads to the spheres. The pressure 608 is applied through set screws to lock ball and socket joints in their position.

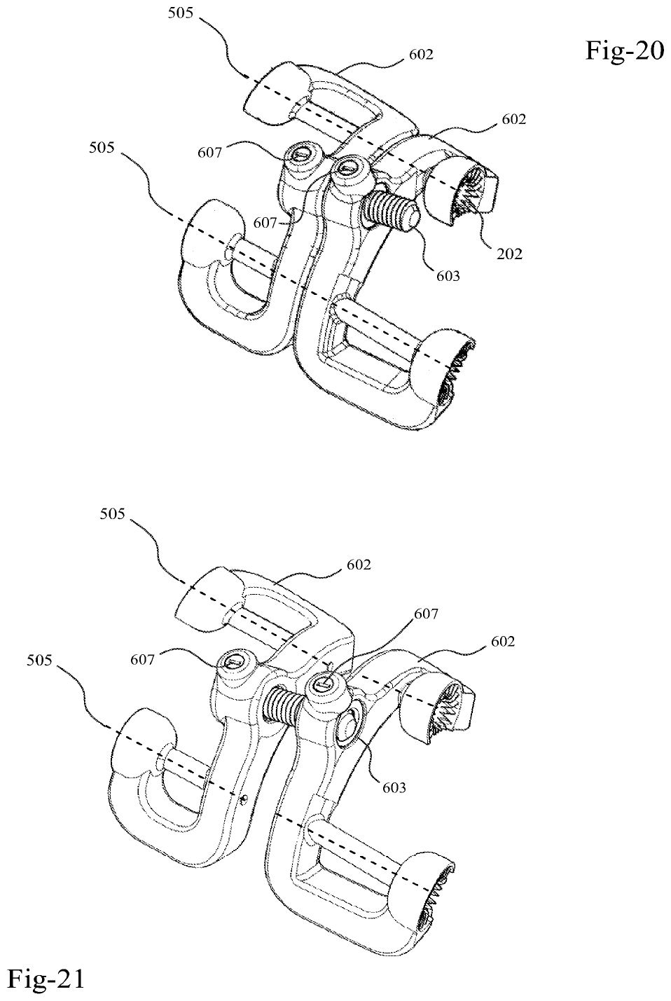

FIG. 20 illustrates the threaded fasteners with ball and socket joints and two further divided scaffold assemblies with radial spline locking surfaces and a spherical socket 602 of FIG. 19 in closed configuration.

FIG. 21 illustrates the threaded fasteners with ball and socket joints and two further divided scaffold assemblies with radial spline locking surfaces and a spherical socket 602 of FIG. 19 in expanded configuration.

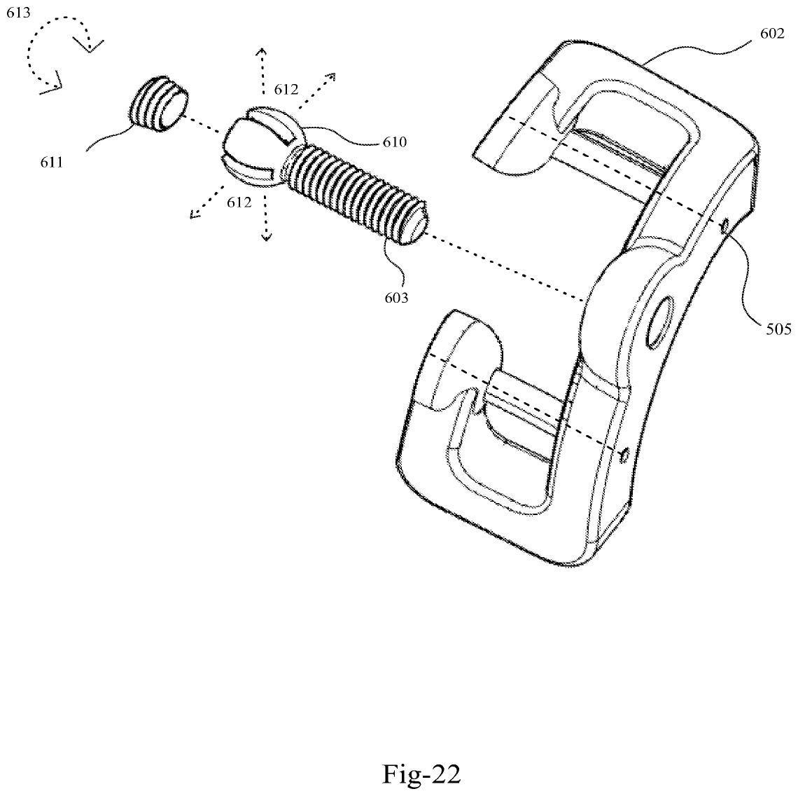

FIG. 22 illustrates an additional method in the form of a locking mechanism between the ball joint and its socket. The method consists of a slightly modified sphere 610 section of a threaded connector and an additional conical screw to incorporate a living hinge type mechanism. The sphere 610 is cut into slices from the top, allowing the structure to expand outwards 612 when a conical setscrew 611 is inserted 613. The outwards expansion of the sliced sphere 610 creates extra friction within the socket of further divided scaffold assembly with radial spline locking surfaces thus stabilizing the 3D alignment. Axial tethers 505 are passing through further divided scaffold assembly with radial spline locking surfaces and spherical socket 602 in accordance with the principles of the invention.

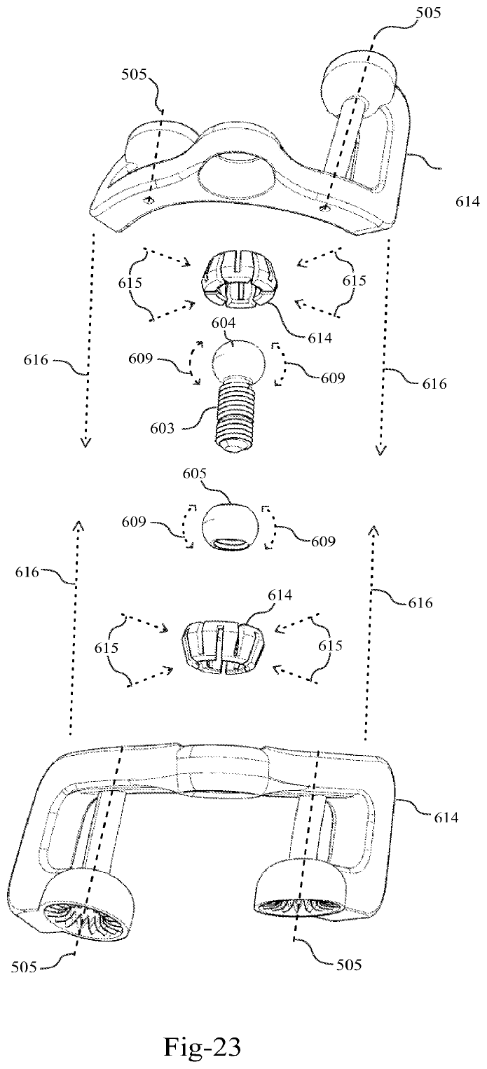

FIG. 23 illustrates an additional method in the form of a locking mechanism between the ball joint and it's socket by introducing additional collets 614 between the spherical outer shells and their sockets. In this method the 3D position of threaded connectors and axially adjacent divided scaffold assemblies is stabilized with the grip 615 from the collets griping spherical outer shells of 604 and 605. The further divided scaffold assembly with radial spline locking surfaces and spherical socket 602 is modified to accommodate conical outer surfaces of collets is demonstrated with 614. In its essence the method is converting the axial tension from axial theaters 616 to constrain the 3D alignment between axially adjacent split cells of the body scaffold.

With all the methods described, the body scaffolds are becoming increasingly complex and difficult to apply in real life conditions. Also, adjustments to the tension from axial tethers are increasingly used for stabilizing the body scaffolds thus requiring a practical solution. Present invention introduces a rotary ratchet embedded to the orthotic for locking, releasing, and adjusting the tension of the axial tethers with ease.

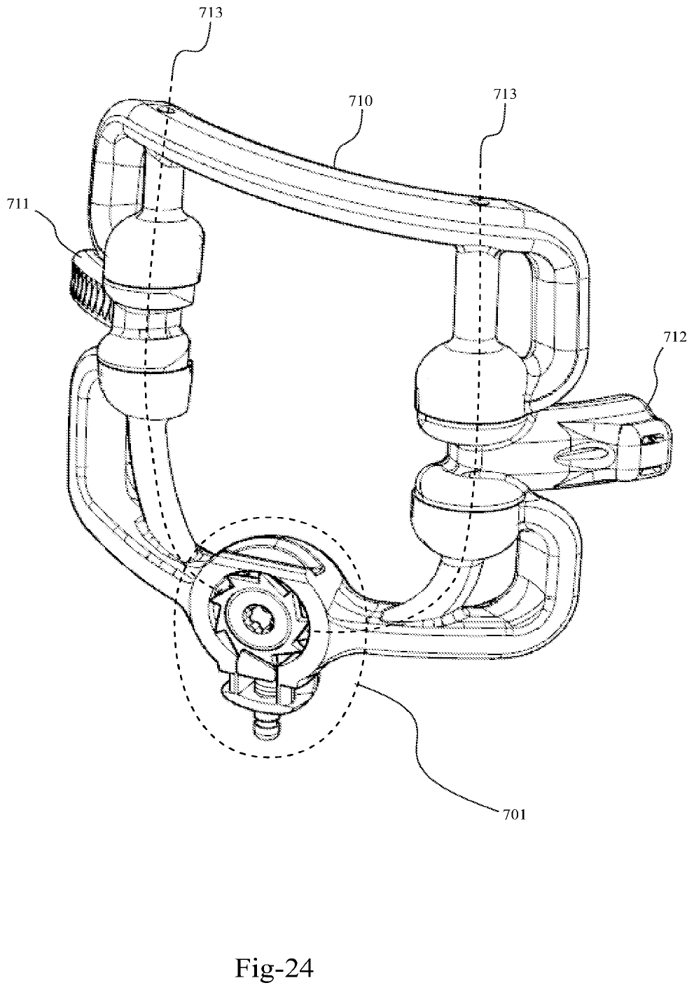

FIGS. 24-28 illustrate a rotary ratchet and pawl mechanism 710 embedded to the proximal end of an axially and circumferentially split scaffold cell. Rotary ratchet mechanism contains a housing element, a rotary ratchet with a V-shaped groove spanning across the gear, a spring-loaded pawl with a quick release and additional nut and bolt style locking for fixing the axial tether and a cap.

FIG. 24 illustrates a single axially and circumferentially split cell is modified with an embedded rotary ratchet 710, a pivoting circumferential male connector with spline locks 711, a pivoting circumferential female connector with spline locks 712, and an axial tether coupled with a rotary ratchet 713.

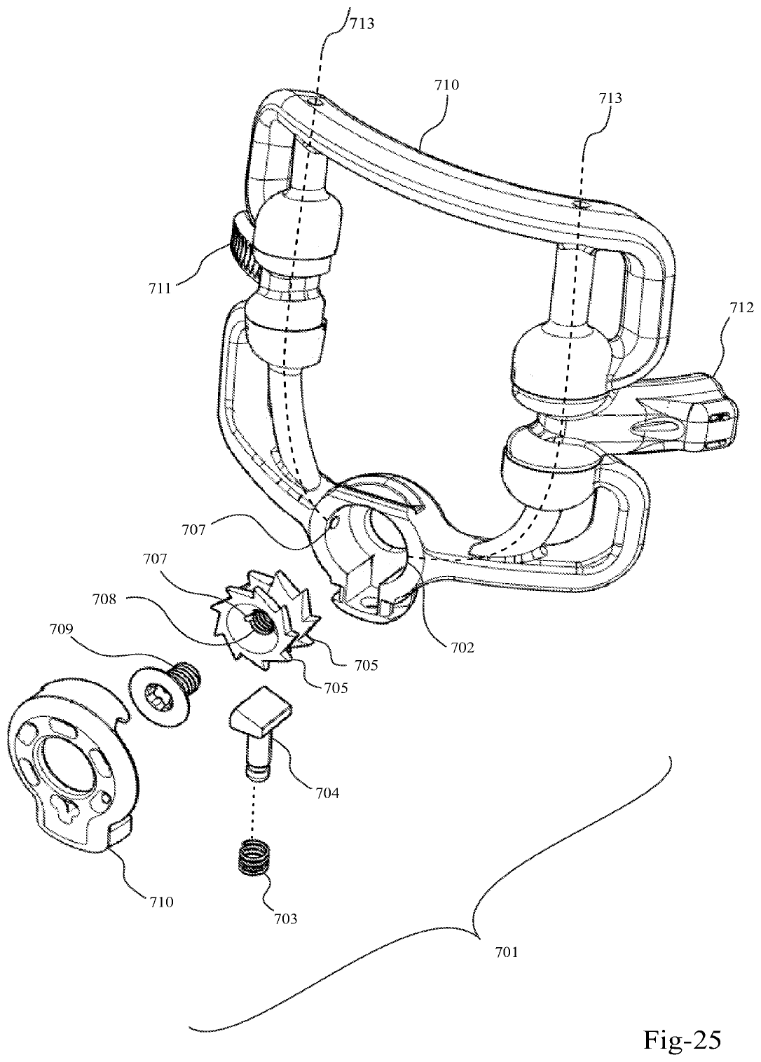

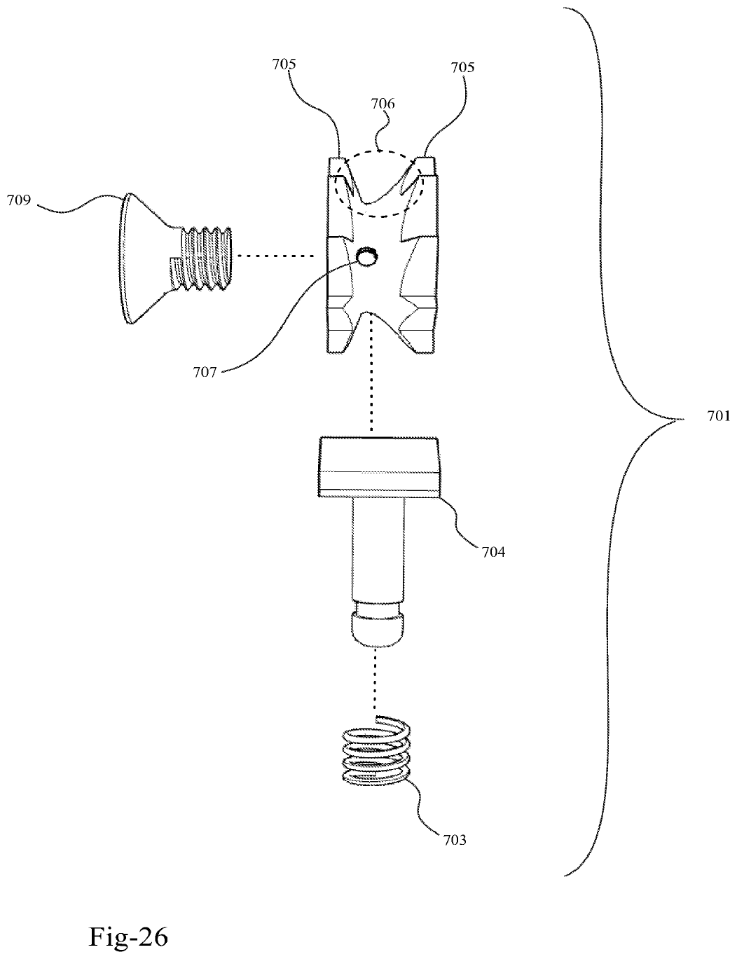

FIGS. 25 and 26 is providing greater detail to the rotary ratchet 710 of FIG. 24 in blowup demonstrations. The rotary ratchet comprises a rotary gear with teeth allowing movement in one direction 705 while having a V-shaped groove 706 for accommodating excess axial tether. The axial tether passes through the V-shaped groove to the center of the rotary gear through openings 707 on the surface. The tether is fixed to the rotary gear by tightening a bolt 709 to the tethered socket 708 of the rotary gear 705, squeezed by both the bolt 709 and tethered socket 708. The rotary ratchet also comprises a fitting pawl 704 for allowing movement in the same direction. The pawl 704 also is fitted with a pillar for assembling a spring 703 to enable quick release function to the rotary ratchet.

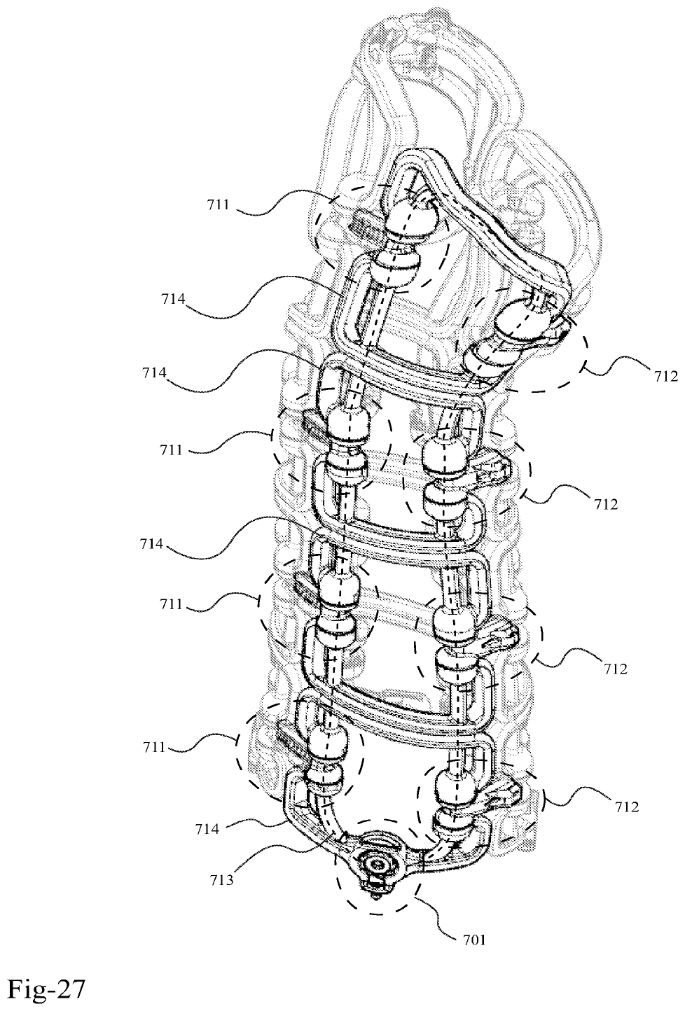

FIG. 27 is an embodiment of an extra adaptive orthosis with four longitudinally split segments and four circumferentially split segments comprising a total of sixteen individual body scaffold cells held together with pivoting slip joints with radial gears, and four axial tethers coupled with four rotary ratchets. An axially split segment of the extra adaptive splint is highlighting three individual body scaffold cells 714 and one modified scaffold cell with an embedded rotary ratchet 710 individual cell is held together with four male type 711 and four female type 712 pivoting circumferential connectors with radial spline locks, and an axial tether coupled with a rotary ratchet 713 is looping through the embodiment.

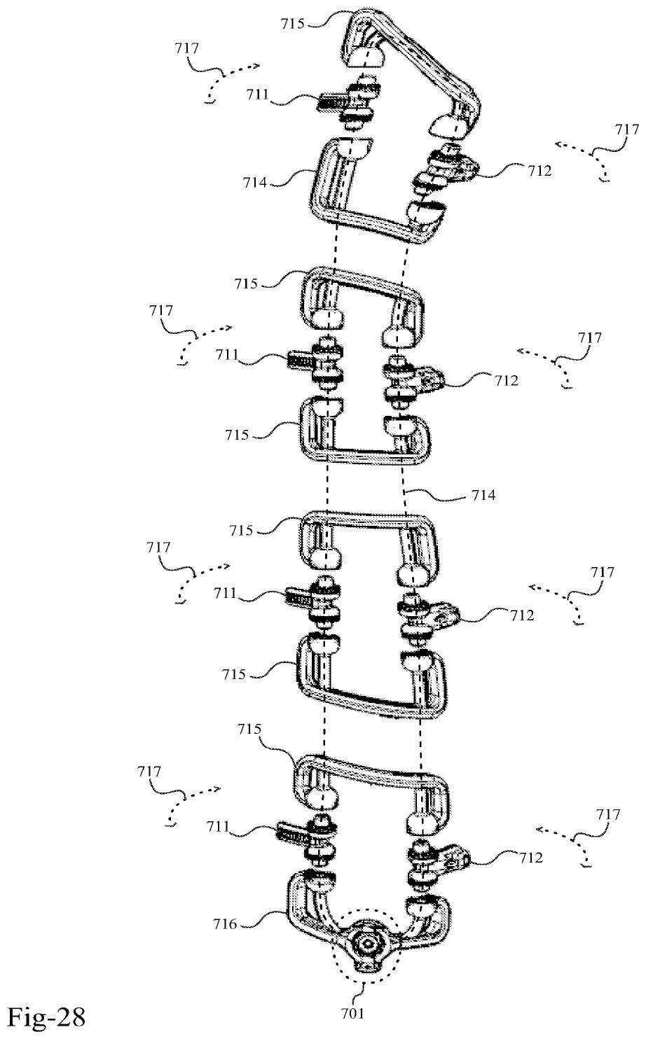

FIG. 28 is an exploded view of the axially split segment of the extra adaptive orthosis of FIG. 27 with seven further divided scaffold cell assemblies with radial spline locking surfaces 715, with one further one divided scaffold cell assembly with radial spline locking surfaces and embedded rotary ratchet 716, one rotary ratchet mechanism 701 and an axial tether coupled with a rotary ratchet 713 is looping through the embodiment. The adjusted position 717 of 6 pivoting circumferential connectors can now be constrained simultaneously with just tightening the rotary ratchet, significantly easing the adjustment process.

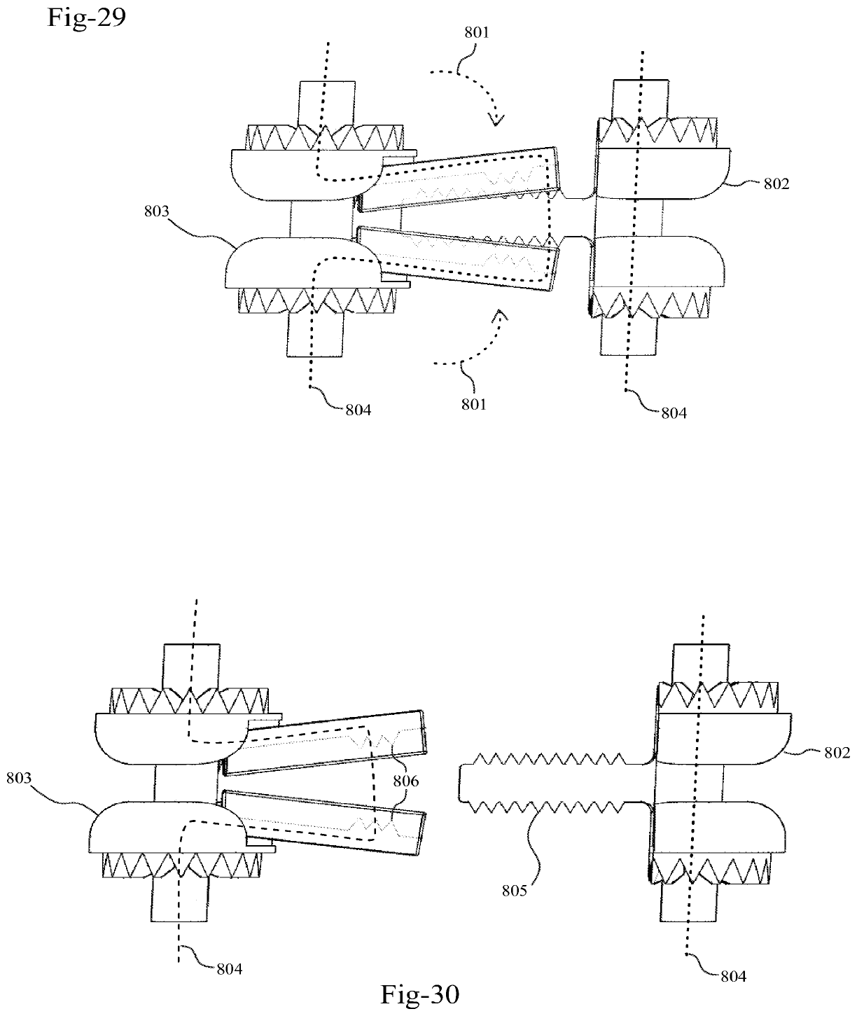

Taking advantage of the rotary ratchet mechanism 710; a more practical circumferentially expanding slip joint mechanism is described between FIGS. 29-31. The locking mechanism is configured to create a clamping grip 801 between circumferentially expanding connectors. The method relies on the tensional force from the rotary ratchet to lock both connectors together. The circumferential connectors are configured to have interconnecting teeth and convenient geometric opening to allow the passage of the axial tethers.

FIG. 29 illustrates a pivoting and clamping circumferential slip joint with radial spline surfaces in expanded and open configuration from front view. A male type pivoting circumferential connector with radial spline surfaces and clamping teeth is illustrated with 802. A female type pivoting circumferential connector with radial spline surfaces and clamping teeth is illustrated with 803. The clamping effect 801 is achieved by increasing the tension of the axial tether coupled with a rotary ratchet 804. The clamping teeth of both connectors are ghosted in the illustration.

FIG. 30 illustrates a male 802 and female 803 type pivoting circumferential connector with radial spline surfaces and clamping teeth in spaced apart configuration. The clamping teeth of male connector is illustrated with 805 and matching female clamping teeth 806 to provide greater detail.

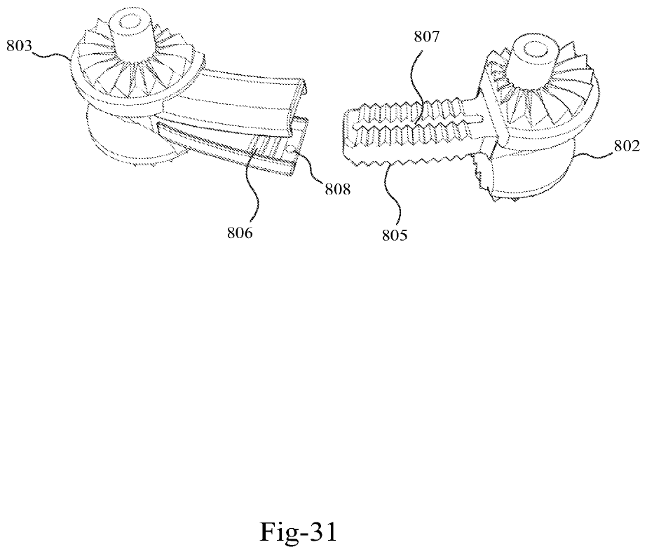

FIG. 31 illustrates male 802 and female 803 type pivoting circumferential connectors of FIG. 30 in 3D demonstration. The slot spanning along the length of male connector's clamping teeth is illustrated with 807 and the entrance point of the axial tether to the circumferential female connector is illustrated with 808.

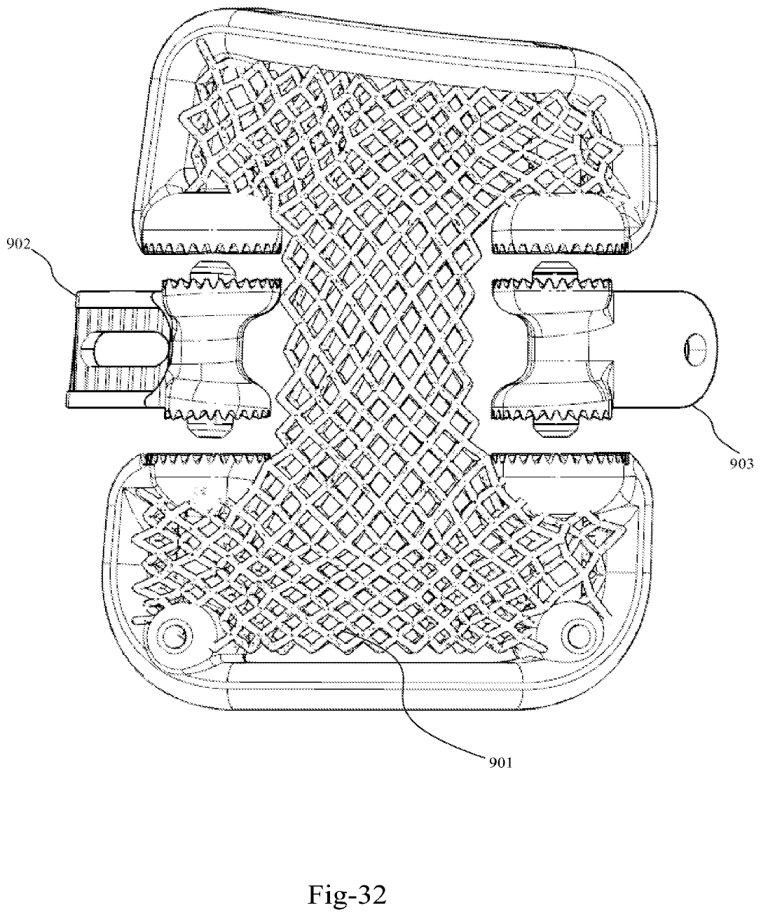

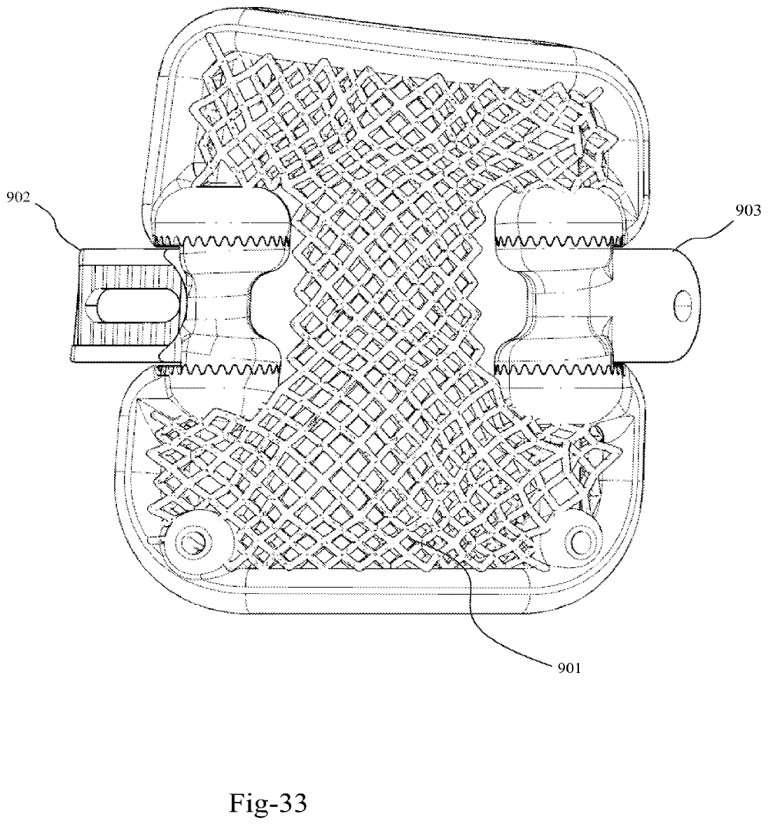

In order to further ease the fitting and assembly processes of axially and radially split body scaffolds, the present invention introduces auxetic lattice geometries spanning across split cells. FIG. 32 illustrates an axially and radially split cell with lattice spanning across its length 901 in expanded configuration and FIG. 33 is showing the same cell with lattice spanning access its length in closed configuration. Pivoting circumferential connectors with radial spline locks and axial tethers are included in the illustration according to the principles of the invention. The method incorporates a single segment or cell with a configured auxetic lattice, capable of stretching and compressing axially in tandem with exterior forces. FIG. 17 demonstrates the lattice in expanded state easing assembly and reducing parts. FIG. 18 demonstrates the lattice in a compressed state.

The foregoing embodiments are presented by way of example only; the scope of the present invention is to be defined by the following claims.

What is claimed is:

1. A conformable orthosis comprising:
a scaffold having a longitudinal axis and being configured to be removably placed over a body surface, wherein the body scaffold is divided into a plurality of scaffold cells having first and second sides and tops and bottoms, wherein the first and second sides on circumferentially adjacent scaffold cells are separable along axial lines and the tops and bottoms on axially adjacent scaffold cells are separable from each other along circumferential lines; and
a first circumferential connector pivotally attached to the first side of at least some of the scaffold cells and a second circumferential connector pivotally attached to the second side of at least some of the scaffold cells;
wherein the first and second circumferential connectors are configured to detachably connect at an adjustable distance therebetween; and
wherein the tops and bottoms on axially adjacent scaffold cells are adjustably coupled to each other.

2. A conformable orthosis as in claim 1, further comprising axial tethers which adjustably couple the tops and bottoms on axially adjacent scaffold cells.

3. A conformable orthosis as in claim 2, wherein the axial tethers pass axially through a pivotal axis of at least some of the first and second circumferential connectors.

4. A conformable orthosis as in claim 2, further comprising at least one uptake spindle for adjustably tightening one or more axial tethers.

5. A conformable orthosis as in claim 4, wherein the uptake spindle comprises a rotary ratchet and pawl mechanism.

6. A conformable orthosis as in claim 2, further comprising spacers configured to be placed between the tops and bottoms of axially adjacent scaffold cells.

7. A conformable orthosis as in claim 1, wherein axially adjacent scaffold cells are connected by threaded fasteners.

8. A conformable orthosis as in claim 7, wherein the threaded fasteners are incorporated into ball and socket joints to permit realignment of the axially adjacent scaffold cells.

9. A conformable orthosis as in claim 1, wherein the first and second circumferential connectors comprise rotating spindles with radially extending coupling tabs.

10. A conformable orthosis as in claim 9, wherein the coupling tabs of the first and second circumferential connectors are configured to detachably lock with the coupling tabs of the second and first circumferential connectors, respectively, of circumferentially adjacent scaffold cells.

11. A conformable orthosis as in claim 9, wherein at least some of the radially extending coupling tabs have grooved surfaces configured to be locked with grooved surfaces on coupling tabs on adjacent scaffold cells.

12. A conformable orthosis as in claim 9, wherein the rotating spindles have upper and/or lower radially splined surfaces configured to selectively lock with the scaffold cell.

13. A conformable orthosis as in claim 1, wherein the first and second circumferential connectors further comprise driving screws configured to engage rotary gears on a periphery of at least some of the rotating spindles to rotate the spindles to adjust an angle of the coupling tabs.

14. A conformable orthosis as in claim 1, wherein the scaffold is configured to circumscribe a body limb, a body joint, or a body torso.

15. A conformable orthosis as in claim 14, wherein the body scaffold comprises an orthotic aid.

16. A conformable orthosis as in claim 1, wherein the body scaffold comprises a three-dimensional lattice.

17. A conformable orthosis as in claim 16, wherein the three-dimensional lattice was produced by three-dimensional printing using a scan of the body surface as a model.

18. A method for fabricating a conformable body interface, said method comprising:
generating or obtaining a data set which represents a scaffold intended to apply corrective or supportive forces to a three-dimensional, soft tissue body surface;
wherein the scaffold is divided into a plurality of scaffold cells having first and second sides and tops and bottoms, wherein the first and second sides on circumferentially adjacent scaffold cells are separable along axial lines and the tops and bottoms on axially adjacent scaffold cells are separable from each other along circumferential lines;
fabricating based on the data set a three-dimensional scaffold configured to be removably placed over the three-dimensional body surface to conform to said surface, wherein the data set defines at least a first circumferential connector pivotally attached to the first side of at least some of the scaffold cells and a second circumferential connector pivotally attached to the second side of at least some of the scaffold cells.

19. A method as in claim 18, further comprising detachably connecting the first and second circumferential connectors at a selected distance therebetween.

20. A method as in claim 19, wherein detachably connecting comprises rotationally positioning adjacent pairs of coupling tabs.

21. A method as in claim 19, wherein rotationally positioning comprises driving a rotary gear with a driving screw.

22. A method as in claim 20, wherein detachably connecting further comprises engaging grooved surfaces on coupling tabs on circumferentially adjacent scaffold cells.

23. A method as in claim 18, further comprising adjustably coupling the tops and bottoms on axially adjacent scaffold cells to each other.

24. A method as in claim 23, wherein adjustably coupling the tops and bottoms on axially adjacent scaffold cells to each other comprises applying tension to axial tethers which span axially adjacent scaffold cells of the scaffold.

25. A method as in claim 24, wherein applying tension to the axial tethers comprises ratcheting ends of the tethers.

26. A method as in claim 23, further comprising placing spacers between axially adjacent scaffold cells to adjust an axial length of the scaffold.

27. A conformable body interface produced by the method of claim 18.
